(19) United States
(12) Patent Application Publication
(10) Pub. No.: US 2011/0275092 A1
(43) Pub. Date: Nov. 10, 2011

(54) METHOD FOR DETECTING BIOLOGICAL MARKERS BY AN ATOMIC FORCE MICROSCOPE

(75) Inventors: Kongxin Hu, Beijing (CN); Liping Zhang, Beijing (CN); Ruijin Ping, Beijing (CN)

(73) Assignee: CHINESE ACADEMY OF INSPECTION AND QUARANTINE, BEIJING (CN)

(21) Appl. No.: 13/129,422

(22) PCT Filed: Dec. 31, 2008
(86) PCT No.: PCT/CN08/73882

§ 371 Date: Jun. 22, 2011

(30) Foreign Application Priority Data
Nov. 18, 2008 (CN) 200810226849.5
Dec. 31, 2008 (CN) PCT/CN2008/073882

(51) Int. Cl. G01N 33/53

(52) U.S. Cl. 435/7.1

(57) ABSTRACT

A method for detecting biological markers involves preparing sample slices using a hard granular labeling material such as hard nano-gold granular material. The sample slices are fixed to sample patches. The sample is scanned using the atomic force microscope (AFM) in tapping mode to collect the height, amplitude and phase data of the hard granular material. The hard labeling material is mainly determined through changes in discrepancies in phase diagram color, while height and amplitude diagrams are used to provide auxiliary evidence of sample morphological features. Integrating these data with the state of the biological target object can thus determine the existence of a marked object.

### FIELD OF THE INVENTION

The invention relates to the field of detecting biolabels using scanning probe microscopes and the related scanning probe mode sensors, especially the method for carrying out the detection of biolabels by atomic force microscope (AFM).

### BACKGROUND OF THE INVENTION

At present, the biolabeling technique is widely applied in the biological field. The basic principle is to use the signal functions of labels combined with the specific binding attributes of biological materials and further indirectly reflect the existence of biological binding role through the labels' signals. Usually, a bio-interaction such as the antigen-antibody binding reaction is difficult to be observed by the naked eye when the reaction is insufficient or the antigen is half-antigen, or the antibody is a univalent antibody. But by biolabeling techniques and some certain specific physical or chemical instruments, the interaction signals can be detected easily. Typical examples are the enzyme-labeled immunoassay (EIA), the immunological fluorescence assay (IFA), the radioactive immunoassay (RIA) and the like. Based on the high specificity of the antigen-antibody binding, free biolabels and bound biolabels can be separated through washing, chromatography or the like; the antigen-antibody interaction can be identified through enzymatic reaction, fluorescence signals, and radioactivity and so on. The labeling substance can be conjugated to an antibody, antigen molecules or other ligands, so it can track the existence of the ligands. In this way, it can amplify and transform the reactions, which can not be seen by the naked eye, to something visible, detectable and traceable, such as light, color, electrical, pulse and other signals through chemical or physical measures. The established technique, also known as the biolabeling technique, is based on combining the specificity of bio-interaction and the sensitivity of labeled molecules detection. The technique can be used for detecting antigens or antibodies; it is more specific and accurate than that of the conventional serological techniques. The technology is widely applied in the immunological field, and can also be extended to labeling detection of nucleic acids, receptors, ligands and other biological substances.

The biological labeling technique can also be combined with the micro-imaging technique in order to accomplish the positioning analysis of the morphology of sample and labels at the nano-scale level. The immunoelectron microscopy (IEM) technique has achieved the distinguishing of the biological background materials with the characteristic of electronic dense of gold nanoparticles. The immunoassay of nano-scale virus particles and other biological substances can be accomplished by a transmission electronic microscope (TEM). However, at present, immunoelectron microscopy is still the only virus identification method which simultaneously combines the viral high-resolution micro-morphology with the specific immunological recognition technique. Although IEM is a destructive morphological technique which requires dyeing and fixation and is not applicable for automated detection, the technique still remains the “reference” method in terms of identification of non-cultivable virus. The core reason is that the specificity of immunology can be accurate to the binding position of a single virus.

As the development of the high-resolution morphology detection technique must depend on the emergence of a novel high-resolution instrument, to a certain extent, the development of new substitute technique for IEM is limited. Along with the emergence of the novel high-resolution observation tool-atomic force microscope (AFM), due to its advantage of observing biosamples at physiological conditions, the AFM seems the ideal tool for observing biological samples, and the simple sample preparation and the high-resolution three-dimensional imaging mode enable the AFM to directly investigate the nanoscale virus particles by size, roughness and other quantified indexes without fixation, dyeing or other destructive sample preparation methods, thus the AFM has become an important tool for studying the topographic features of biosamples. As the imaging principle of probe-scanning of the AFM is significantly different from IEM, the AFM has been one of the most important developments in the surface imaging instrumentation fields over the recent ten years. The AFM can detect the biosamples in natural, which leads the samples' morphology to be closer to the physiological state, so that it can not only observe the local micro-region morphology of the biological samples but also can detect the interaction force among biological molecules as well as study the structural and functional relationships, and investigate the microbe's recognition field.

Based on the high-resolution imaging capacity and the advantage of the biological application of the AFM, if the AFM is combined with the specific biolabeling detection technologies, it will became another novel substitute technique which is similar to immune electron microscopy detection. Thereby, it will combine the high-resolution micro-imaging with the specificity of biolabel recognition, and will expand the scope of the AFM in the biological application field.

### SUMMARY OF THE INVENTION

The invention is aimed at providing a method for carrying out biolabel detection by direct AFM scanning. The method combines the micro-high-resolution imaging performance of the AFM with the specificity detection performance of the biolabeling methods in the biological field. The method can be directly used for immunodiagnosis of labels and positioning analysis of microbes and other biological materials.

The method of carrying out biolabel detection by using direct AFM is shown in the invention, which is specifically as follows:
1) Hard granular marking material as label material (such as nanogold, nanosilver, glassmilk and so on) is prepared by publicly known means and conjugated with biological material (such as an antibody) on the hard granular material, to obtain the biolabeled hard particles;
2) Soak and clean a sample carrier coverslip by a washing solution, then wash it with ultrasonic waves, clean it in the deionized water and carry out surface treatment with poly-L-lysine;
3) Biological sample is directly dripped on the surface of the sample carrier coverslip, and is uniformly spread. After incubating in a wet box, wash it in deionized water, and then blow-dry it with nitrogen;
4) BSA (bovine serum albumin) solution is dripped to cover the surface of the sample carrier coverslip; incubate it in the wet box, wash it in deionized water, and then blow-dry with nitrogen;
5) Solution of the biolabeled hard particles is dripped to cover the surface of the sample carrier coverslip. After incubating in the wet box, wash it in deionized water, and then blow-dry it with nitrogen;
6) The sample carrier coverslip is investigated on the AFM;
7) The tapping mode is used to scan the sample at room temperature, and simultaneously collect data of height, amplitude and phase of the sample;
8) Comparing with the morphologic consistency of height, amplitude and phase images comprehensively and taking the local phase contrast in phase image as main judgment for the existence of hard particles and taking the height and amplitude images as auxiliary judgment for morphological features and positioning of the targeted biosamples;
9) The presence of biorecognition is further identified, followed by analysis of the extent of the hard labeling material particles beside or on the targeted biosample.

Further, the hard particle as label material is gold nanoparticle with hard attribute.

Further, in step 2) the specific process is as follows: the solution of step 2 is washed ultrasonically in washing solution for 30 min, further flush it in running deionized water for 2 min, wash it ultrasonically in deionized water for 15 min, take it out, blow-dry it with nitrogen, further treat it with poly-L-lysine for 5 min, and blow-dry it with nitrogen. Further, the washing solution in step 2) comprises of 3.5% sulfuric acid, 12% AES, 12% LD-650 and 1.2-1.5% sodium chloride, and the PH of the washing solution is adjusted to 7-8 using NaOH solution.

Further, in step 3) the specific process is as follows: the sample carrier coverslips are coated with the biological sample, incubated in the wet box at 37° C. for 1 h, washed in deionized water three times for 5 min each time, and finally blow-dried with nitrogen.

Further, in step 4) the specific process is as follows: 10% of BSA solution is dripped to cover the surface of the sample carrier coverslip, and then incubated in the wet box at 37° C. for 1 h, washed in deionized water three times for 5 min each time, and finally blow-dried with nitrogen.

Further, in step 5) the specific process is as follows: solution of the hard labeling material particles is dripped to cover the surface of the sample carrier coverslip, and then incubated in the wet box at 37° C. for 1 h, washed in deionized water three times for 5 min each time, and finally blow-dried with nitrogen.

Further, in step 8) the combined judgment is carried out by observing the brightness data characteristics of the phase image in combination with height and amplitude morphologic feature data.

Further, the probe used in the tapping mode of the AFM in step 7) is an RTESP probe produced by silicon, the length of a cantilever is 115-135 μm, the elastic constant k is 20-80 N/m, the resonance frequency is 200-400 kHz, the curvature radius of a probe point is 5-10 nm, and the tapping frequency of the probe is 1 Hz.

The biological sample carriers in the method are washed by washing solution and treated by the poly-L-lysine. The obtained carrier has good adsorptive and flat surface, and it is applicable to the preparation of most of the AFM biological sample sheets. The method for preparing sample carriers has the advantage of using common reagents, low price and simple treatment. The method does not use concentrated sulfuric acid, strong base and other strong corrosive reagents to carry out sectional timing treatment, so that the above method greatly shortens the total time. The invention uses the AFM tapping mode to scan the biological material and hard labels with different hardness. The phase image is mainly used to reflect the different softness and hardness of the labeling particles and the biological material and it can reflect the presence of biological antigen and antibody and other biological binding role in combination with the morphologic characteristics of the height image and the amplitude image by the analytical way of collecting three signals at the same time, so the method can carry out positioning detection analysis.

### BRIEF DESCRIPTION OF THE DRAWINGS

FIG. 1 is the optimal protein detecting curve for nanogold conjugation;

FIG. 2A shows the AFM scanning result of a sample coverslip treated with poly-L-lysine in embodiment 1 of the invention;

FIG. 2B shows the AFM scanning result of a nano-gold sample sheet prepared by the sample coverslip and treated with poly-L-lysine;

FIG. 2C shows the AFM scanning result of the sample sheet of nano-gold conjugated with an antibody and prepared by the sample coverslip pretreated by the poly-L-lysine;

FIG. 3A shows the AFM scanning result of the sample sheet of Escherichia coli with recombinant expression of measles virus nucleoprotein as blank control, which is prepared in embodiment 2 of the invention;

FIG. 3B shows the AFM scanning results of an Escherichia coli blank control sample sheet with combination of antibody-labeled nano-gold and recombinant expression of measles virus nucleoprotein, as prepared in embodiment 2;

FIG. 4A shows the AFM scanning result of an influenza virus sample sheet, as prepared in embodiment 3 of the invention;

FIG. 4B shows the AFM scanning result of an antibody-labeled nano-gold binding with influenza virus, as prepared in embodiment 3.

### DETAILED DESCRIPTION OF THE INVENTION

The invention uses an AFM to scan the sample; Nano-gold is used as a hard granular material label to conjugate with the specific antibody. Using the morphologic scanning and identifying process of the specific antigen-antibody binding on the microbe's surface as an example, the aim is to construct a new method for specific distinction between hard-labeling material signals and biological background material signals via the AFM. However, the method of the invention is not limited to the nano-gold granular material which is taken as the hard label. Nano-metal, nano-compound and other hard labeling materials can also developed to carry out the biological labeling of specific antigen, antibody and nucleic acid. The AFM is used to carry out direct observation and detection and to realize the effective combination of micro-high-resolution imaging performance and specificity detection performance of the AFM in the biological field. Expanding the biological application scope of the AFM, it can be directly used for biolabel immunodiagnosis or positioning analysis of microbes and other biological materials and applied in related hybridization technique labels and other biological detecting fields.

The steps of the invention are as follows:

### I. Preparation of Nano-Gold

### 1. Gold Boiling

Sulfuric acid was used to soak a conical beaker for 24 h. Took it out, washed it in water for 10 times and washed it in distilled water 8 times, then washed it in double distilled water 6 times. The conical beaker and a stirring rod used for gold boiling were only used for nano-gold. The well washed conical breaker was filled with 100 ml double distilled water. Firstly, place it on a heating magnetic stirring machine to heat to boiling, optimized the rotating of stirring rod for preventing liquid from spilling. 1 ml of chloroauric acid was added and then 1.5 ml of disodium citrate was added. The color change of the solution was observed from colorless to black and wine red, and then cooled it when the color was stabilized at the wine red, and loaded it in a brown bottle at 4° C.

### 2. Adjustment of PH Value

Prepare 15 ml of well prepared nano-gold solution in a small conical flask (specially used for nano-gold), then the PH value of the nano-gold solution was adjusted to 8.5-9.0 using 1 mol/L of K₂CO₃.

### 3. Determination of Optimal Protein Dose

2 mmol/L of borate buffer solution (PH9.0) was dripped to dilute the antibody till the amount of the antibody is 0.2 mg/ml. According to Table 1, gradual steps were operated by the diluted antibody and other related reagents.

TABLE 1
Minimal Protein Dose of Nano-gold Determined by using Spectrophotometer

| | Test Tube No. | | | | | | | | | | |
|---|---|---|---|---|---|---|---|---|---|---|---|
| Reagent | 1 | 2 | 3 | 4 | 5 | 6 | 7 | 8 | 9 | 10 | control |
| Nano-Gold | 1.0 | 1.0 | 1.0 | 1.0 | 1.0 | 1.0 | 1.0 | 1.0 | 1.0 | 1.0 | 1.0 |
| (ml) | | | | | | | | | | | |
| Buffer (μl) | 0 | 10 | 20 | 30 | 40 | 50 | 60 | 70 | 80 | 90 | 100 |
| Antibody | 100 | 90 | 80 | 70 | 60 | 50 | 40 | 30 | 20 | 10 | 0 |
| (μl) | | | | | | | | | | | |

Shake and stand for 2 min

| | | | | | | | | | | | |
|---|---|---|---|---|---|---|---|---|---|---|---|
| 10% NaCl | 100 | 100 | 100 | 100 | 100 | 100 | 100 | 100 | 100 | 100 | 100 |
| (μl) | | | | | | | | | | | |

Shake and stand for 10 min for observation of Minimal Protein Dose

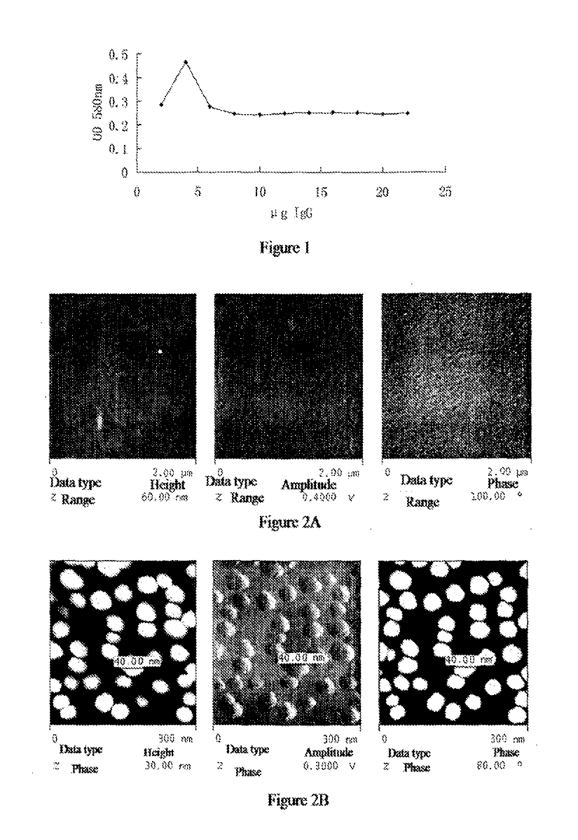

The OD value was used as a longitudinal coordinate and the protein dose was used as a transverse coordinate for making the curve shown in FIG. 1. When the curve was close to the transverse axis, the protein dose at the point was the minimal stable protein content. The protein was further added 20% of dose on the above basis, then the actual dose of stable nano-gold labeling protein was finally obtained. According to FIG. 1, we can see that the actual dose of the protein used for labeling 1 ml of nano-gold solution was 9.6 μg in the experiment.

### 4. Labeling of Nanogold

The optimal protein dose was added into the 1 ml of the nano-gold, mixed and stood for 5 min.

### 5. Washing of Nanogold

10% of BSA (bovine serum albumin) was added, and mixed, shook up, stood for 10 min and centrifuged at 12000 r/min at 4° C. for 30 min; discarded the supernatant, then resuspended in 1 ml Re-suspension (0.01 mol/LTris TBS pH8.2, including 1 BSA, flicked the precipitated nano-gold, uniformly mixed it, and further centrifuged it at 12000 rpm/min at 4° C. for 30 min; Next, discarded the supernatant, added 500 μl of Re-suspension, flicked the precipitated nano-gold, then uniformly mixed it, and further centrifuged it at 1000 r/min for 10 min at 4° C. A 0.2 μm filter was used to filter and remove aggregates. The labeled nano-gold was obtained finally.

### II. Preparation of Carrier Coverslip

A circular sample carrier coverslip with the diameter of 15 mm was placed into washing solution for carrying out washing ultrasonically for 30 min. The washing solution contains 3.5% of sulfuric acid, 12% of AES, 12% of LD-650 and 1.2-1.5% of sodium chloride, and NaOH solution was used for adjusting the PH value to 7-8. The washing solution may also be routine detergent such as washing powder and the like. Further, the remaining washing solution was flushed away in running deionized water for 2 min. 50 ml of deionized water was used to wash ultrasonically for 15 min, then take it out, and blow-dried it with nitrogen, then treat it with poly-L-lysine for 5 min. The coverslip should have no sticking phenomenon when taken out and blow-dried. When it is treated with the poly-L-lysine, the coverslip should be immersed into the solution completely to ensure the uniform surface treatment of the coverslip.

### III. Preparation of Sample Carrier Coverslip

Respectively, the gold nanoparticle sample and protein-conjugated gold nanoparticle sample or microbial sample were diluted with 1×PBS, dripped 50 μl of sample solution to coat uniformly on the sample carrier coverslip after the treatment by the poly-L-lysine. Next, it was incubated at 37° C. for 1 h, washed in deionized water three times for 5 min each time, and blow-dried with nitrogen. 10% of BSA (bovine serum albumin) solution was dripped to cover the surface of the sample carrier coverslip for blocking, then it was incubated in the wet box at 37° C. for 1 h, and washed in deionized water three times for 5 min each time. 50 μl of the prepared gold nanoparticles with the well labeled corresponding antibody was dripped to cover the surface of the sample carrier coverslip, then it was incubated at 37° C. for 1 h, washed with 1×PBS five times for 1 min each time, washed with deionized water three times for 5 min each time, and blow-dried with nitrogen. The operation should be gentle when adding the nano-gold, liquid should completely cover the surface of the microbial sample as far as possible, but the pipette tips should not touch the microbial sample when adding the liquid. Tweezers should not touch the surface of the coverslip with the sample when moving the sample. The sample was placed into a culture dish after being scanned, and the culture dish was well sealed by a sealing film for preserving it at 4° C.

### IV. The AFM Investigation

The prepared gold-labeled microbial sample was fixed on a specific 15 mm circular patch by using a double-sided adhesive tape, and the tapping mode of the AFM was used to scan the sample. AFM images were obtained through a VECCO Multimode and a NanoScopeIIIa controller at room temperature and atmospheric conditions with E type scanner, the used probe was RTESP probe produced by silicon. The length of a cantilever was 115-135 μm, the elastic constant was 20-80 N/m, the resonance frequency was 200-400 kHz, the curvature radius of probe point was 5-10 nm, and the tapping frequency of the probe was 1 Hz. The measurement mode was a constant force mode. Image output signals were height, amplitude and phase. The results were analyzed by adopting the comprehensive comparison of the three images and by using the local color changing of different phase image as the main judging basis, and by using the height image and amplitude image as the assistant judging basis for the morphological feature of biological object the existence of a labeled object was determined.

### Embodiment 1

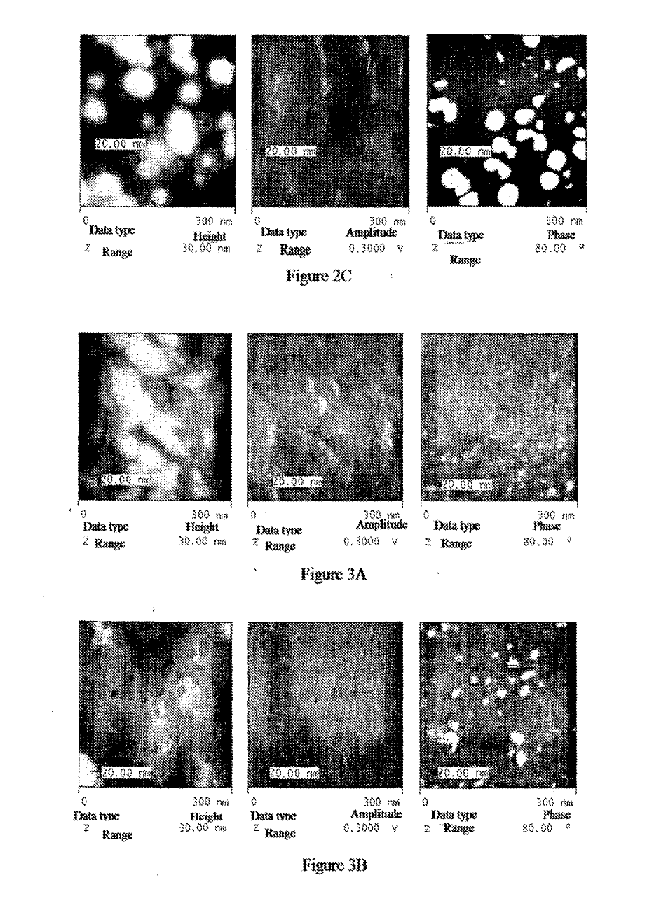

A circular sample carrier coverslip with a diameter of 15 mm was placed in washing solution (including 3% of sulfuric acid, 3% of AES, 0.4% of sodium hydroxide and 1.2-1.5% of sodium chloride). We washed it ultrasonically for 10 min. Then took it out, blow-dried it with nitrogen, treated it with poly-L-lysine for 5 min, and dried it naturally. The coverslip specially used for AFM on a circular patch was fixed, The sample was scanned using the tapping mode of the AFM and the image obtained is shown in FIG. 2A: the background of the sample sheet was observed as uniform and the surface fluctuation was less than 1 nm, porous structures on surface were also observed which might be easy to adsorb the sample in the physical adsorption way, and the hardness shown as phase contrast seemed similar in each region of the treated coverslip.

Dripped 50 μl of nano-gold solution on the surface of the coverslip pretreated with the poly-L-lysine, and incubated the coverslip at 37° C. for 30 min. Washed it five times by 1×PBS and 1 min for each time, washed it in deionized water three times and 3 min for each time, then blow-dried it with nitrogen. The coverslip was pasted on a circular patch. Next, the sample was scanned at tapping mode of the AFM. The image obtained is shown in FIG. 2B. The unlabeled gold particles were regular, and most of them were circular shape, the diameters of them were about 20 nm, and the morphologic contour and the size of the gold particles in the three signal collection images displayed highly consistency by observation of the height, amplitude and the phase images comprehensively. The contrast of brightness and darkness between the gold particles and the background in the phase image was easily observed. Generally, the contrast of brightness and darkness in the phase image reflected the softness and hardness, and the viscosity and other properties on the surface of the sample. As for the gold particles, the hardness was its main property here, so the brightness stands for hardness and the darkness stands for softness in the phase image, then the obvious contrast of brightness and darkness indicates the differences between the hardness of the gold particles and soft background biomaterial.

The chart of the antibody-conjugated gold nanoparticles is shown in FIG. 2C. The significant differences between FIG. 2C and FIGS. 2 A and 2 B are as follows:

The morphologic contour of the particles observed from the phase image in FIG. 2 C showed obvious inconsistency with that observed in the height and the amplitude image. The situation disappearance or the irregular nano-particles were common. Compared with FIG. 2A and FIG. 2B, it can be seen that the irregular gold particles in the phase image of FIG. 2C are brighter, while the other missing parts seem merged in biological background objects. Therefore, the irregular brighter regions show the presence of the gold nanoparticles, the “disappeared” parts of nanoparticles could indirectly indicate the coverage region of immunoglubin, which must be the main position coated with the protein. The amplitude image was used for assisting in further defining the morphologic features of the antibody-conjugated nanogold and the targeted sample. Therefore, the irregular bright region in the phase image could be used to definitely indicate the differentiation characteristic of the hard gold nanoparticles and the targeted biomaterial and other background biological substances. And then it further shows that the labeling particles can be used as biolabels for signal collection and judgment for AFM immunoassay.

### Embodiment 2

A recombinant engineering cell strain BL-21-30a-MVn expressing the measles virus nucleoprotein (the cell strain was from Exotic Disease Transmission Room of China Academy of Inspection and Quarantine) was inoculated on LB solid medium for culturing less than 24 hours at 37° C. Single colony was picked up with a pipette tip and transferred into the LB liquid medium, then shaken in a constant temperature shaking-cabinet (37° C., 265 rad/min) overnight. 1 ml of bacterial liquid was transferred into a 1.5 ml tube, and centrifuged at 3000 rpm for 10 min, and the supernatant was discarded. Next 1 ml of 1×PBS was used to suspend bacterial for sedimentation, and then centrifuged at 3000 rpm for 10 min. The above steps were repeated three times. Finally the washed bacterial cells were suspended in 500 μl of 1×PBS. 50 μl of bacterial liquid was dripped, evenly on two 15 mm circular coverslips pretreated with poly-L-lysine, respectively, and then incubated it at 37° C. for 30 min. Filter paper was used to absorb away the remained liquid from the coverslips. The sample sheet was washed 5 times with 1×PBS for 1 min each time, then washed in deionized water 3 times for 3 min each time, blow dried with nitrogen. This sample sheet was taken as the control sample. 50 μl of the prepared gold nanoparticles labeled with antibody against measles virus nucleoprotein was added to the other sample sheet, incubated at 37° C. for 30 min, washed with 1×PBS 5 times for 1 min each time, washed in the deionized water 3 times for 3 min each time, and blow-dried with nitrogen. Fixed the coverslip on a circular patch and scanned the sample at the tapping mode on AFM.

Signals were collected though the three modes of height, amplitude and phase. The obtained image are shown in FIG. 3A, which displayed a local surface micro-region picture of recombinant Escherichia coli BL-21. The image displays a regular morphology, partial depression of the bacteria, smooth surface, no cyst-like bulges, and visible regular particles on its surface. No obvious contrast of brightness and darkness was seen on the phase image, the general performance displayed as dark as a soft material, and highly consistency was shown in the morphologic contour of the height and the amplitude image (scanning range 300 nm×300 nm). FIG. 3B is a picture of the BL-21-30a-MVn treated with antibody-labeled nanogold against the measles virus nucleoprotein. Compared with the image of the untreated bacteria control sample (FIG. 3A), the local surface, displays slight deformation with irregular fluctuations and visible layering or pits. On the phase image, irregular bright points of different sizes are observed, and the bright points appear obvious accumulation in some area of the bacterial surface. Further analysis with the amplitude and the height image indicate the morphologic features of obvious granules' sediment on the bacterial surface, thereby the presence of label particles on the bacterial surface can be determined.

### Embodiment 3

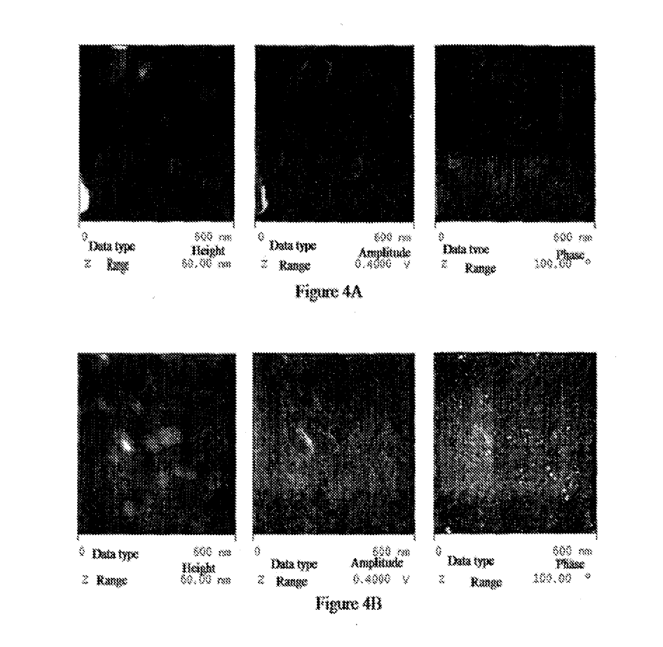

The purified influenza virus (A1 strain) was prepared from sucrose dense gradient centrifuge after ultra-filtration and concentration of its chick embryo culture. 50 μl of the purified virus solution was evenly spread on a 15 mm circular coverslip pretreated with poly-L-lysine, and then incubated at 37° C. for 30 min. Filter paper was used to absorb away the liquid from the surface of the coverslip. The sample sheet was washed with 1×PBS 5 times for 1 min each time, then washed in deionized water 3 times for 3 min each time, blow-dried with nitrogen. The sample sheet was used as control. Another sample sheet was prepared as above. 50 μl of the prepared nano-gold conjugated with the antibody against influenza virus was added to this sample sheet. This sample sheet was incubated it at 37° C. for 30 min, washed with 1×PBS 5 times for 1 min each time, then washed in the deionized water 3 times for 3 min each time, and blow-dried with nitrogen. The coverslips are fixed on special circular patches with the diameter of 15 mm for AFM scanning, and the tapping mode of the AFM was used to scan the samples. FIG. 4A shows the images of purified influenza virus morphology and displays uniform distribution of virions, spherical and regular shape with diameter about 80-120 nm. High consistency of the morphologic contour was seen among the height, amplitude and phase image, and no obvious bright and dark contrast were observed in phase image (scanning range of 600 nm×600 nm). FIG. 4B shows the scanning results of the influenza virus treated with antibody-conjugated nanogold. Compared with the control FIG. 4A (without gold-labeled antibody treated), FIG. 4B shows that the morphology of virus particles was also clear and visible. Some pits are observed on some virion surfaces on the amplitude image, while the pits are displayed as higher brightness contrast on the phase image and indicate the binding sites of the labeling gold nanoparticle (scanning range 600 nm×600 nm), thereby reflecting the binding sites of the antibody-conjugated goldparticles with the surfacial antigen of influenza virus.

Through a series of investigations of biological materials, gold nanoparticle materials and the combination of both with the AFM, the invention provides a novel marker-signal recognition method mainly from phase data of AFM, which is based on the different texture or softness and hardness of the scanned materials, achieving differentiation of the specific label signal of the hard granular materials from the biological background materials. Being different from the traditional biomarker signal collection mode of immunoassay such as IEM, the AFM uses a scanning probe as the signal collection tool. This label recognition method greatly expands the range of biological applications of the AFM, and leads the immune atomic force microscopy to the practical application stage. Because the hard labels do not need to be limited to nano-gold, the labels provided here can be further extended to include plastic balls, glass balls, other nano-metal particles or synthetics and other hard granular materials.

Generally, the invention provides a novel and intuitive marker-signal interpretation method via comprehensive analysis of the existence of “brighter spots” on the phase image. Distributional characteristics of viral shapes appear on the height and amplitude images, which results in differentiation of specific signaling mode of the immunogold materials from the biological background materials. The method opens up a distinctive method for AFM to integrate the micro-high-resolution imaging performance and specificity detection of antibody, and realizes specific differentiation between the hard labeling materials and the biological background materials under AFM scanning. As specific bio-recognition and morphological images can be obtained at the same time during this process and the biomarker methodology is applicable with any ligands, such as the specific antigen, antibody, nucleic acid and others, the AFM can be further employed for direct observation and identification of viruses and probably used for recognizing and positioning analysis of many types of biomaterials on a nanometer scale.

1. The invention is using the method of the detection of biolabel by AFM, as follows:
1) Hard granular material was prepared by the publicly known means, and was labeled with biological material such as antibody, to obtain hard biolabeling particles;
2) A sample carrier coverslip was soaked and cleaned by washing solution, then washed it by ultrasonic waves, cleaned it in deionized water and carried out surface treatment with poly-L-lysine;
3) Biological sample material was directly dripped on the surface of the sample carrier coverslip, uniformly smeared it. After incubating in a wet box, washed it in the deionized water, and then blow-dried it with nitrogen;
4) BSA (bovine serum albumin) solution was dripped to cover the surface of the sample carrier coverslip for blocking purpose, incubated it in the wet box, washed it in the deionized water, and then blow-dried it with nitrogen;
5) Solution of the hard labeling material particles was dripped to cover the surface of the sample carrier coverslip. After incubating in the wet box, washed it in the deionized water, and then blow-dried it with nitrogen;
6) The sample carrier coverslip was fixed on a sample patch arranged on the AFM;
7) The AFM tapping mode was used to scan the sample under the conditions of room temperature and atmospheric environment; and simultaneously collected data of height, amplitude and phase of the scanned sample;
8) Comprehensively compare the morphologic consistency of the height, amplitude and phase images, taking the local higher brightness spots (phase contrast) in phase image as main judgment for the hard granular labels existence and taking the height and amplitude images as auxiliary judgment for the targeted biosample morphology and positioning to determine the existence of the labeled object.
9) Further determine the presence of a labeled object after determining the hard labeling material particles.

2. The method of carrying out the biolabel detection by using the AFM according to claim 1, characterized in that the hard granular material was gold Nanoparticle material with hard attribute.

3. The method of carrying out the biolabel detection by utilizing the AFM according to claim 1, characterized in that the treatment process in the step 2 is specifically as follows: ultrasonic washing was carried out in the washing solution for 30 min, further flushed it in the running deionized water for 1 min, and carried out the ultrasonic washing in the deionized water for 15 min, took it out, blow-dried it with nitrogen, further use the poly-L-lysine to treat for 5 min, and blow-dried it with nitrogen.

4. The method of carrying out the biolabel detection by utilizing the AFM according to claim 1, characterized in that the washing solution in the step 2) comprises 3.5% of sulfuric acid, 12% of AES, 12% of LD-650 and 1.2-1.5% of sodium chloride, and NaOH solution is used for adjusting PH value till the PH value is 7-8.

5. The method of carrying out the biolabel detection by utilizing the AFM according to claim 1, characterized in that the treatment process in the step 3 was specifically as follows: the sample carrier coverslip coated with the biological sample material was incubated in the wet box for 1 h at 37° C., washed it in the deionized water three times for 5 min each time, and finally blow-dried it with nitrogen.

6. The method of carrying out the biolabel detection by utilizing the AFM according to claim 1, characterized in that the treatment process in the step 4 was specifically as follows: 10% of BSA solution was dripped to cover the surface of the sample carrier coverslip, then incubated it in the wet box at 37° C. for 1 h, washed it in the deionized water three times for 5 min each time, and finally blow-dried it with nitrogen.

7. The method of carrying out the biolabel detection by utilizing the AFM according to claim 1, characterized in that the treatment process in the step 5 was specifically as follows: the solution of the hard labeling granular material was dripped to cover the surface of the sample carrier coverslip, then incubated it in the wet box at 37° C. for 1 h, washed it in the deionized water three times for 5 min each time, and finally blow-dried it with nitrogen.

8. The method of carrying out the biolabel detection by utilizing the AFM according to claim 1 in the step 8), characterized in that the combined judgment is carried out by using the brightness morphologic data characteristics of the phase image in combination with height and amplitude morphologic data.

9. The method of carrying out the biolabel detection by utilizing the AFM according to claim 1, characterized in that the probe used in the tapping mode of the AFM in the step 7) is an RTESP probe produced by silicon, the length of a cantilever is 115-135 μm, the elastic constant k is 20-80 N/m, the resonance frequency is 200-400 kHz, the curvature radius of a probe point is 5-10 nm, and the tapping frequency of the probe is 1 Hz.
